(19) United States
(12) Patent Application Publication
(10) Pub. No.: US 2020/0205720 A1
(43) Pub. Date: Jul. 2, 2020

(54) SYSTEMS, DEVICES, AND METHODS FOR ACQUIRING, VALIDATING AND ANALYZING ATHLETIC MOVEMENT DATA

(71) Applicant: SPARTA SOFTWARE CORPORATION, Menlo Park, CA (US)

(72) Inventor: Phillip Patrick Wagner, Menlo Park, CA (US)

(21) Appl. No.: 16/733,353

(22) Filed: Jan. 3, 2020

Related U.S. Application Data
(63) Continuation of application No. PCT/US18/40687, filed on Jul. 3, 2018.
(60) Provisional application No. 62/528,866, filed on Jul. 5, 2017.

(51) Int. Cl. A61B 5/00; A63B 71/06

(52) U.S. Cl. CPC A61B 5/4023; A63B 2230/62; A63B 71/0622

(57) ABSTRACT

Systems, devices and methods are provided for acquiring, validating and analyzing athletic movement data. Generally, a sensor device, such as a force plate, is provided for sensing certain characteristics of an athletic movement. A local computing device coupled to the sensor device can be configured to receive sensor data indicative of the characteristics of the athletic movement, process and extract information from the sensor data, and transmit the processed sensor data to a remote server system. The remote server system can be configured to store, aggregate and update the processed sensor data in a database, and can also generate one or more normalized scores correlating to the athletic movement. The normalized scores can indicate to a user a susceptibility to injury, progression towards return to play or propensity for success with respect to a particular sport.

### CROSS-REFERENCE TO RELATED APPLICATIONS

The subject application is a continuation of PCT Patent Application No. PCT/US18/40687, filed Jul. 3, 2018, which claims priority to U.S. Provisional Patent Application No. 62/528,866, filed on Jul. 5, 2017, both of which are incorporated by reference herein in their entirety for all purposes.

### FIELD

The subject matter described herein relates generally to systems, devices, and methods for acquiring, validating and analyzing athletic movement data. In particular, sensor data for a user performing an athletic movement is captured by one or more sensors, validated, and transformed into one or more normalized scores. The normalized scores are based at least in part on population data residing in a database on a remote server system. The normalized scores can indicate the user's susceptibility to injury, progression towards return to play or suitability for a particular sport.

### BACKGROUND

Advances in kinesiology and sensor technology have enabled researchers to capture and study vast amounts of data regarding the human body during movement, and in particular, during athletic movement. Some researchers, for example, have utilized electromyography (“EMG”) for detecting and recording electrical activity produced by skeletal muscles during a golf swing. Other researchers have employed near-infrared spectroscopy (“NIRS”) to measure and monitor oxygenation in muscle and other tissues during cycling exercises. Still other researchers have tried affixing inertial sensors, or inertial measurement units (“IMUs”), to athletes during speed and agility exercises to quantify certain biomechanical metrics.

Despite an abundance and diversity of data, however, a significant challenge remains in translating the information gathered in these studies and others into actionable results. For example, every year the National Football League hosts the Combine, an event in which athletes are run through a battery of technical tests that purport to predict an athlete's likelihood of success as a football player. Athletes and agents spend significant amounts of money to prepare for the Combine, which utilize high-technology sensors and other devices. Yet, a five-year study of the NFL Combine results for 306 football players concluded that there was no consistent statistical relationship between the Combine tests and professional football performance. In another study of basketball players in 2012, researchers were able to show a link between better performance and certain physical characteristics, such as a stiffer torso or a longer standing long jump. However, the study was unable to identify any meaningful patterns with respect to injury resilience.

Thus, needs exist for systems, devices and methods for acquiring, validating and analyzing athletic movement data, and in particular, for the purpose of determining an athlete's susceptibility to injury, progression towards return to play or suitability for a particular sport.

### SUMMARY

Provided herein are example embodiments of systems, devices and methods for acquiring, validating and analyzing athletic movement data. Generally, a local computing device is provided, where the local computing device is communicatively coupled to one or more sensors that are adapted to sense various characteristics of one or more athletic movements. These characteristics can include, for example, a plurality of ground reaction forces. The one or more sensors can be included within a single housing, such as that of a force plate. The local computing device, to which the one or more sensors are coupled, can also include, amongst other components, communications circuitry, one or more processors and a memory coupled to the one or more processors. The memory is configured to store instructions that, when executed by the one or more processors, cause the one or more processors to perform various method steps for acquiring, validating and analyzing athletic movement data. For example, the local computing device can be configured to receive and process sensor data indicative of the various characteristics of the one or more athletic movements, and in turn, transmit the processed sensor data to a remote server system.

A remote server system is provided for receiving and storing the processed sensor data, and can also be configured for transmitting back to the local computing device one or more normalized scores correlating to the processed sensor data associated with the one or more athletic movements. The normalized scores can be T-scores, which are normalized by various factors, such as by body weight, by gender, by preferred sport or by preferred position within a sport. In addition, the remote server system can include a database comprising stored processed sensor data indicative of characteristics of various athletic movements for a population of athletes. In this regard, and as described in further detail below, the normalized scores can provide a variety of actionable indicators to an athlete such as, for example, susceptibility to injury, progression towards return to play, or suitability for a particular sport, to name a few.

To ensure the integrity of the acquired and processed sensor data, several data validation methods are also provided. For example, in some embodiments, prior to the user performing an athletic movement, the measured weight of the user is compared to a stored reference weight. If a weight mismatch is detected, e.g., if the measured weight is inaccurate or the user has misidentified herself, then the user is instructed to weigh in again. In other embodiments, one or more predetermined weight thresholds are monitored during the athletic movement which can detect, for example, a user prematurely stepping off the force plate, or the user not landing on the force plate with sufficient force. In still other embodiments, a final data check is performed before the processed sensor data is transmitted to the remote server system, which can be used to detect a corrupt file. These and other data validation methods are described in further detail below.

These embodiments and others described herein are improvements in the fields of computer-assisted biomechanics and, in particular, in the area of computer-based kinetic and kinematic analysis. Other systems, devices, methods, features and advantages of the subject matter described herein will be apparent to one with skill in the art upon examination of the following figures and detailed description. The various configurations of these devices are described by way of the embodiments which are only examples. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of one or more local computing devices each of which can be coupled to a sensor device, a network, and a remote server system including a database.

FIG. 2 is a block diagram of an example embodiment of a local computing device.

FIG. 3 is a block diagram of an example embodiment of a remote server system.

FIGS. 4A and 4B are flow chart diagrams depicting example embodiment methods for assessing a user's static stability.

FIGS. 5A and 5B are pictorial diagrams depicting certain steps in the example embodiment methods of FIGS. 4A and 4B.

FIGS. 6A and 6B are flow chart diagrams depicting example embodiment methods for assessing a user's dynamic stability.

FIGS. 7A and 7B are pictorial diagrams depicting certain steps in the example embodiment methods of FIGS. 6A and 6B.

FIGS. 8A to 8C are example embodiments of graphical user interfaces for displaying various characteristics of athletic movements.

FIG. 9 is a flow chart diagram depicting an example embodiment method for generating an athletic signature of a user, including data validation steps.

FIG. 10 is a flow chart diagram depicting an example embodiment method for assessing a user's static stability, including data validation steps.

FIG. 11 is a flow chart diagram depicting an example embodiment method for assessing a user's dynamic stability, including data validation steps.

FIGS. 12A to 12D are example embodiments of graphical user interfaces for displaying various data validation notifications.

FIG. 13 is an example embodiment of a graphical user interface for inputting data validation settings.

### DETAILED DESCRIPTION

Before the present subject matter is described in detail, it is to be understood that this disclosure is not limited to the particular embodiments described herein, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

Generally, embodiments of the present disclosure include systems, devices, and methods for acquiring, validating and analyzing athletic movement data. Accordingly, many embodiments can include one or more sensor devices coupled to one or more local computing devices, wherein the one or more sensor devices are configured to measure various characteristics of an athletic movement performed by a user. In addition, many embodiments can include a remote server system which can include, or be communicatively coupled with, a database configured to store processed sensor data associated with various athletic movements for a population of athletes.

In some embodiments, for example, a force plate can be configured to measure a resultant sway velocity associated with a user standing in a balance pose on the force plate. The resultant sway velocity is transmitted to a remote server system, and, subsequently, one or more normalized scores correlating to the resultant sway velocity are displayed on the local computing device. In these embodiments, the normalized scores can reflect a user's static stability.

In other embodiments, a force plate can be configured to measure a peak force and time to stabilize within a predetermined percentage of a reference weight associated with a user jumping from a stationary position to a landing position on the force plate. The peak force and time to stabilize are transmitted to the remote server system and, subsequently, one or more normalized scores correlating to the peak force and time to stabilize are displayed on the local computing device. In these embodiments, the normalized scores can reflect a user's dynamic stability.

Additionally, the present disclosure also includes systems and methods for validating the data acquired by the one or more sensors, and can include, for example, a weight mismatch process, a weight deviation process, a peak force deviation process, a premature end condition monitoring process, and a final data check process, among others, each of which is described in further detail below. The embodiments disclosed herein can include local computing devices, each of which is in communication with a remote server system that is location-independent, i.e., cloud-based. Those of skill in the art will also appreciate that the embodiments disclosed herein can also include local computing devices, each of which is in communication with a remote server system that is located on the same premise and/or local area network as the one or more local computing devices. In these embodiments, the remote server systems which are located on the same premise and/or local area network as the one or more local computing devices can also be configured to synchronize a database containing processed sensor data associated with a population of athletes with a database residing on, or coupled with, a centralized remote server system that is location-independent, i.e., cloud-based.

Furthermore, for each and every embodiment of a method disclosed herein, systems and devices capable of performing each of those embodiments are covered within the scope of the present disclosure. For example, embodiments of sensor devices, local computing devices, and remote server systems are disclosed and these devices and systems can each have one or more sensors, analog-to-digital converters, one or more processors, memory for storing instructions, displays, storage devices, communications circuitries (for wired and/or wireless communications), and/or power sources, that can perform any and all method steps, or facilitate the execution of any and all method steps.

The embodiments of the present disclosure provide for improvements over prior modes in the field of computer-based kinetic and kinematic analysis. These improvements can include, for example, optimization of computer resources, improved data accuracy and improved data integrity, to name only a few. In a number of embodiments, for example, instructions stored in the memory of a local computing device (e.g., software) can cause one or more processors of the local computing device to process and extract certain characteristics from sensor data associated with one or more athletic movements received from a sensor device (e.g., a force plate), and transmit the processed sensor data to a remote server system. Subsequently, the remote server system receives and stores the processed sensor data, and returns to the local computing device one or more normalized scores correlating to the athletic movement. The normalized scores can be T-scores, for example, and displayed on the local computing device as an easy-to-read vertical bar chart. The sensor data on the local computing device can be subsequently discarded. Thus, according to one aspect of the embodiments, memory and hard drive space are conserved at the local computing device because sensor data need not be permanently stored. Likewise, the remote server system need only store processed sensor data (i.e., extracted values), and is not required to process or store raw sensor data, thereby conserving memory, hard drive space and processing power. Thus, computer resources can be significantly conserved both at the local computing device as well as at the remote server system.

The disclosed embodiments also reflect computer-related improvements in data accuracy and data integrity. In some embodiments, for example, the remote server system includes, or is communicatively coupled with a database for storing processed sensor data correlating to a population of athletes. According to one aspect of the disclosed embodiments, the remote server system can be location-independent (i.e., cloud-based), and configured to aggregate processed sensor data from a plurality of local computing devices, which can be located in a plurality of geographically dispersed areas. The remote server system can also provide normalized scores to each local computing system based on the population data contained in the database. The normalized scores can also be can be normalized according to categories, for example, by gender, by body weight, by sport or by position within a sport. By continually aggregating and updating the population data contained within the database, the remote server system can be configured to provide customizable, dynamically generated and accurate scores to the user.

According to another aspect of the disclosed embodiments, improvements in data integrity are also provided through data validation processes during the acquisition of the sensor data. As described in further detail below, the data validation processes can include, for example, a weight mismatch process, a weight deviation process, a peak force deviation process, a premature end condition monitoring process, and a final data check process, among others. Each of these processes, as well as others, are configured to ensure that the acquired sensor data is accurate and correct prior to processing and receiving the processed sensor data by the remote server system.

The improvements of the present disclosure are necessarily rooted in computer-based systems for the acquisition, validation and analysis of athletic movement data, and are directed to solving a technological challenge that might otherwise not exist but for the existence of computer-based kinetic and kinematic analyses. Additionally, many of the embodiments disclosed herein reflect an inventive concept in the particular arrangement and combination of the devices, components and method steps utilized for acquiring, validating and analyzing athletic movement data. Other features and advantages of the disclosed embodiments are further discussed below.

Before describing these aspects of the embodiments in detail, however, it is first desirable to describe examples of devices that can be present within, for example, a system for acquiring, validating and analyzing athletic movement data, as well as examples of their operation, all of which can be used with the embodiments described herein.

### Example Embodiment of System for Acquiring, Validating Analyzing Athletic Movement Data

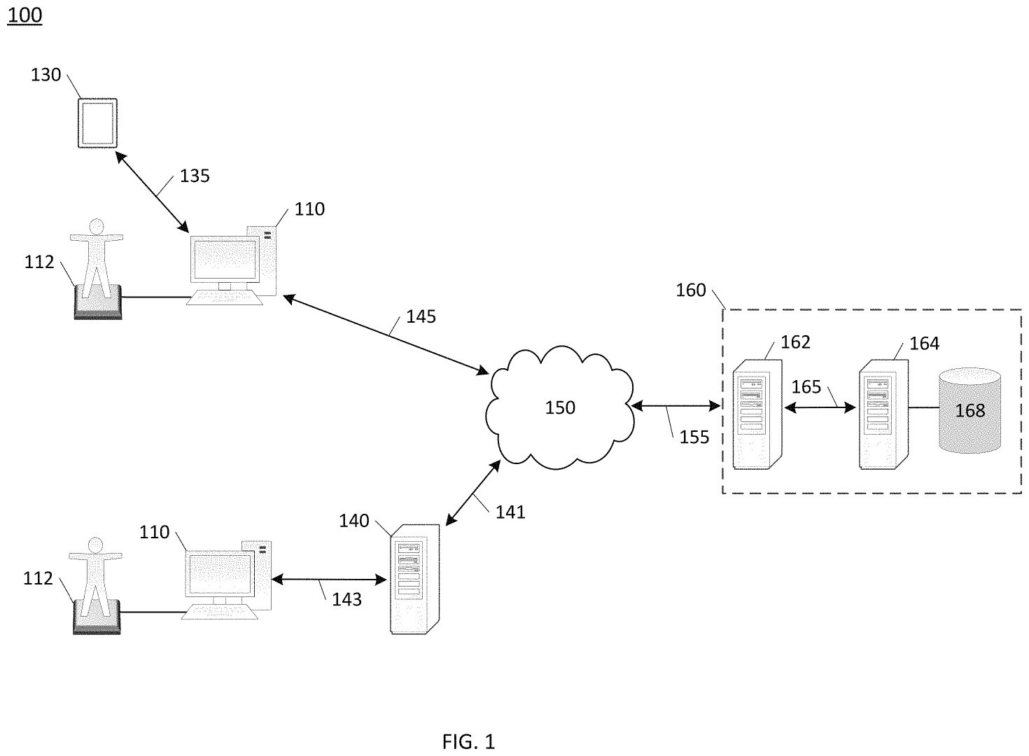

FIG. 1 is a conceptual diagram depicting an example embodiment of a system 100 for acquiring, validating and analyzing athletic movement data, and which can be used with the embodiments of the present disclosure. System 100 includes a remote server system 160 configured to receive data from one or more computing devices 110, and which can comprise a front-end server 162 for interfacing with said computing devices 110, and a back-end server 164 that interfaces with both the front-end server 162 and database 168. Remote server system 160 can be a location-independent server system (e.g., cloud-based), which is accessible by a variety of computing devices 110 in geographically dispersed locations. Front-end server 162 can be in communication with back-end server 164 over a local area network, and can also communicate with computing devices 110 over communication path 155, which can include wired or wireless communications over network 150. In many of the embodiments disclosed herein, network 150 can be the Internet. In other embodiments, however, network 150 can also comprise one or more of a wide area network, a local area network, a metropolitan area network, a virtual private network, a cellular network, or any other type of wired or wireless network. Furthermore, although front-end server 162 and back-end server 164 are depicted in FIG. 1 as two discrete devices, those of ordinary skill in the art will recognize that the functionalities and services of those devices can be implemented on a single centralized device or, in the alternative, can be distributed across multiple discrete devices in geographically dispersed locations. Likewise, those of skill in the art will recognize that the representations of servers in the embodiments disclosed herein, and as shown in FIG. 1, are intended to cover both physical servers and virtual servers (or “virtual machines”).

Referring still to FIG. 1, one or more local computing devices 110 are provided for receiving sensor data from sensor device 112, processing and extracting values from sensor data, and transmitting processed sensor data over network 150 to remote server system 160. Local computing device 110 can be a personal computer, laptop computer, desktop computer, workstation computer, or any other similar computing device, each of which can be communicatively coupled to a sensor device 112, which is configured to sense one or more athletic movements performed by a user. Sensor device 112 can be connected to local computing device 110 via a wired or wireless communication link. Additionally, as shown in FIG. 1, a mobile computing device 130, such as a tablet computer, laptop, smart phone, or wearable computing device, can also be communicatively coupled to local computing device 110 through a wired or wireless communication link. Mobile computing device 130 can be configured to send and receive data to and from sensor device 112 via computing device 110 through communication path 135. In other embodiments, however, mobile computing device 130 can be configured to communicate directly with sensor device 112 through Bluetooth, Bluetooth Low Energy, 802.11x, UHF, NFC or any other standard wireless communications protocol. In some of the embodiments, mobile computing device 130 is configured to operate according to a mobile operating system such as Android and/or IOS. Local computing device 110 can be configured to transmit and receive data over communication path 145 through network 150, which, as described earlier, can comprise the Internet, a wide area network, a local area network, a metropolitan area network, a virtual private network, a cellular network, or any other type of wired or wireless network.

In some embodiments, a local server system 140 can reside on the same local area network as local computing device 110. Local server system 140 can receive and store processed sensor data from local computing device 110, and in turn, transmit locally stored T-scores to local computing device 110 over communications path 143. Local server system 140 can also synchronize a local database with the database 168 of the remote server system 160. In this regard, local server system 140 can serve as a proxy or intermediary between local computing device 110 and remote server system 160. In certain instances, this topology may be preferable, such as where heightened security is needed for local computing device 110 and/or the local area network on which local computing device 110 and local server system 140 reside. For example, the owner of local computing device 110 may not want to permit any or some of the processed sensor data collected through local computing device 110 to be transmitted to the remote server system 168, which may be shared by multiple tenants. Under those circumstances, local server system 140 can serve as a gateway, and conduct one-way synchronization or selective synchronization of the local database with database 168 of remote server system 160.

### Example Embodiment of Local Computing Device

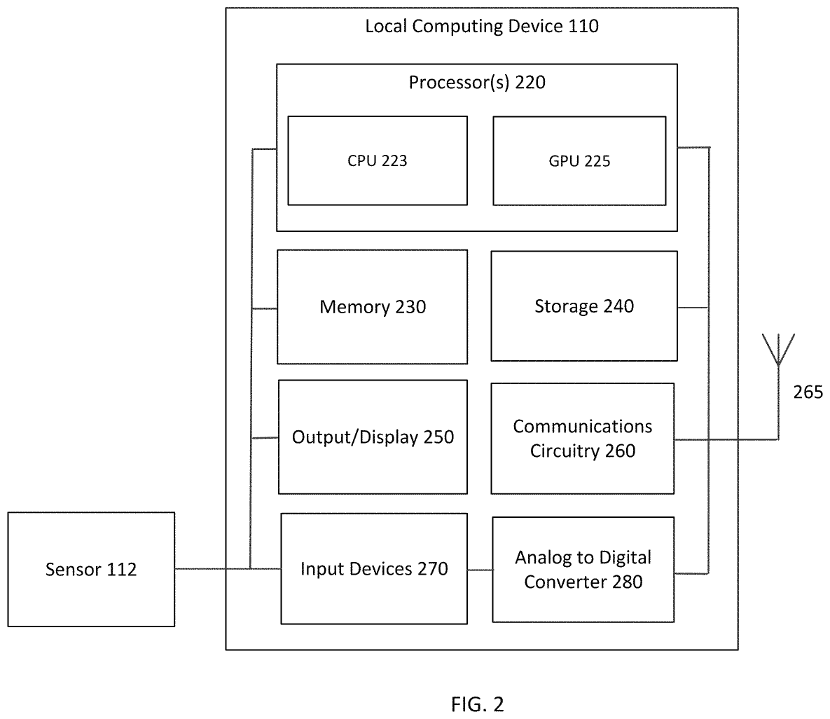

FIG. 2 is a block diagram depicting an example embodiment of local computing device 110. Local computing device 110 can include one or more processors 220, which can comprise, for example, one or more of a general-purpose central processing unit (“CPU”), a graphics processing unit (“GPU”), an application-specific integrated circuit (“ASIC”), a field programmable gate array (“FPGA”), an Application-specific Standard Products (“ASSPs”), Systems-on-a-Chip (“SOCs”), Programmable Logic Devices (“PLDs”), or other similar components. Processors 220 can comprise one or more processors, microprocessors, controllers, and/or microcontrollers, or a combination thereof, wherein each component can be a discrete chip or distributed amongst (and a portion of) a number of different chips, and collectively, can have the majority of the processing capability for acquiring, validating and analyzing athletic movement data. Local computing device 110 can also include memory 230, which can comprise non-transitory memory, RAM, Flash or other types of memory. Furthermore, local computing device 110 can include one or more mass storage devices 240, an output/display component 250, communications circuitry 260, which can include one or more wireless and/or wired network interfaces, an antenna 265 coupled to communications circuitry 260, an analog to digital converter component 280 configured to convert an analog signal received from a sensor device into a digital signal, and an input device component 270, which can include keyboards, mice, trackpads, touchpads, microphones and other user input devices, each of which can be communicatively coupled to local computing device 110 via a wired or wireless connection.

In many of the embodiments of the present disclosure, input devices component 270 can also include a sensor device 112, which can comprise one or more sensors configured to sense various characteristics of an athletic movement. In many embodiments, for example, sensor device 112 can comprise a force plate including one or more piezoelectric sensors within a single housing, wherein the one or more piezoelectric sensors are adapted to measure ground reaction forces while one or more athletic movements are performed by a user. In some embodiments, sensor device 112 can comprise a force plate including one or more strain gauge sensors within a single housing. In still other embodiments, sensor device 112 can include multiple types of sensors, in which data received from a first type of sensor can be used to corroborate the data received from a second type of sensor. For example, sensor device 112 can comprise a force plate including one or more piezoelectric sensors, as described earlier, and additionally, one or more accelerometers embedded within a portion of a user's footwear. Sensor data from the piezoelectric sensors and the accelerometers can be correlated, time synchronized and/or multiplexed by local computing device 110 to determine and corroborate various characteristics of the one or more athletic movements performed by the user. As understood by those of skill in the art, the aforementioned components are electrically and communicatively coupled in a manner to make one or more functional devices.

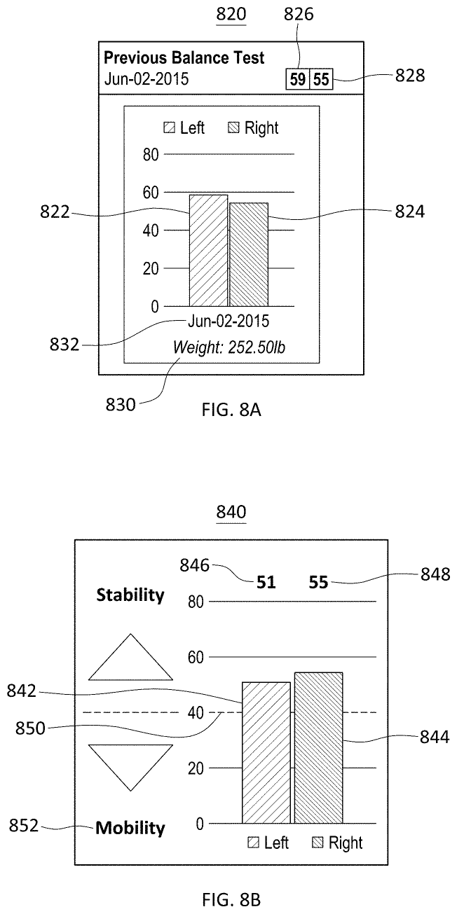

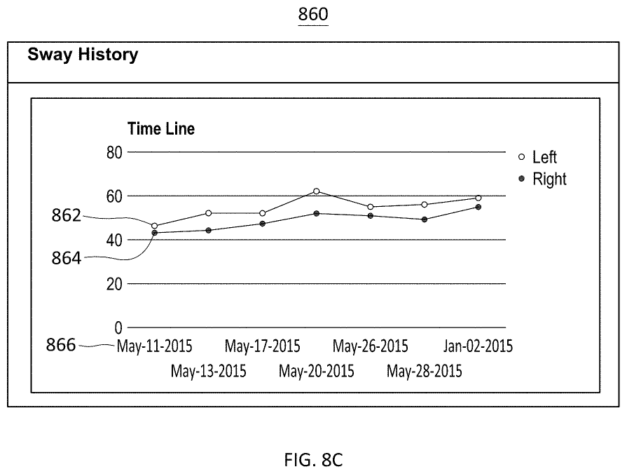

Referring still to FIG. 2, communications circuitry 260 of local computing device 110 can be configured to communicate directly with remote server system 160, or via local server system 140. In many of the embodiments disclosed herein, local computing device 110 is configured to receive sensor data generated by sensor device 112 in response to a user performing one or more athletic moves. The received sensor data can be processed and transmitted to a remote server system which, in turn, transmits one or more normalized scores correlating to the athletic moves performed by the user to the local computing device 110. In many of the embodiment disclosed herein, and as described later with respect to FIGS. 8A to 8C, the normalized scores can be visually displayed through a user interface on local computing device 110. In some embodiments, the normalized scores can be depicted as T-scores in a vertical bar chart (FIGS. 8A and 8B). In other embodiments, the one or more normalized scores can be depicted as a plotted line as a function of time (FIG. 8C). These graphical user interfaces, as well as other visual representations, can be generated by processors 220 in response to instructions, e.g., in the form of a locally installed application, which reside in memory 230 of local computing device 110.

As described earlier, local computing device 110 is represented in FIG. 2 as a personal computer, desktop computer, laptop computer or workstation. However, in some embodiments, the one or more local computing devices 110 can also include laptop computers, tablet computing devices, smartphones, personal digital assistants, wearable computing devices or other mobile computing devices.

### Example Embodiments of Remote Server System

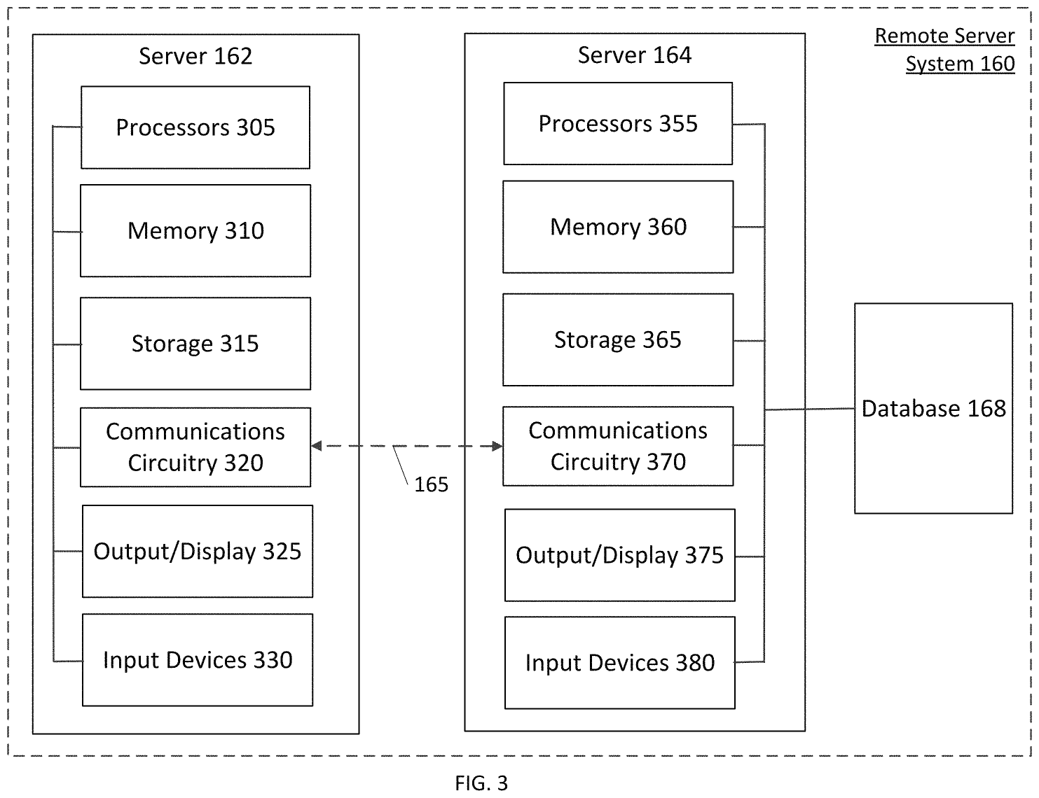

FIG. 3 is a block diagram depicting an example embodiment of remote server system 160 comprising one or more servers, and which can include a front-end server 162 and a back-end server 164. As shown in the diagram, servers 162, 164 can each include, respectively, an output/display component (325, 375), one or more processors (305, 355), memory (310, 360), including non-transitory memory, RAM, Flash or other types of memory, communications circuitry (320, 370), which can include both wireless and wired network interfaces, mass storage devices (315, 365), and input devices (330, 380), which can include keyboards, mice, trackpads, touchpads, microphones, and other user input devices. The one or more processors (305, 355) can include, for example, a general-purpose CPU, a GPU, an ASIC, an FPGA, ASSPs, SOCs, PLDs, and other similar components, and furthermore, can comprise one or more processors, microprocessors, controllers, and/or microcontrollers, each of which can be a discrete chip or distributed amongst (and a portion of) a number of different chips. As understood by one of skill in the art, these components are electrically and communicatively coupled in a manner to make a functional device.

In some embodiments, front-end server 162 can be configured such that communications circuitry 320 provides for a single network interface which allows front-end server 162 to communicate with the one or more local computing devices, as well as back-end server 164. In other embodiments, front-end server 162 can be configured such that communications circuitry 320 provides for two discrete network interfaces to provide for enhanced security, monitoring and traffic shaping and management. In addition, in most embodiments, front-end server 162 includes instructions stored in memory 310 that, when executed by the one or more processors 305, cause the one or more processors 305 to receive processed sensor data from one or more local computing devices, store processed sensor data to a database 168, and generate and transmit one or more normalized scores associated with an athletic movement to a local computing device. In addition, the instructions stored in memory can further cause the one or more processors to perform one or more of the following routines: aggregate processed sensor data by various categories including by gender, by age, by body weight, by preferred sport and/or by position within a preferred sport; generate and store normalized scores associated with an athletic movement for one or more of the aforementioned categories; update normalized scores based on newly received processed sensor data from the one or more local computing devices; and perform synchronization between database 168 and one or more databases residing on local server systems.

Referring still to FIG. 3, server 164 can include database 168 for storing processed sensor data indicative of one or more characteristics of an athletic movement. In some embodiments, database 168 can reside on back-end server 164. In other embodiments, database 168 can be part of a storage area network, for example, to which back-end server 164 is communicatively coupled. Back-end server 164 can also include communications circuitry 370 which can be configured to facilitate communications to and from front-end server 162. Similar to the configuration of front-end server 162, in many embodiments, communications circuitry 370 can include a single network interface, either wired or wireless; or, in other embodiments, communications circuitry 370 can include multiple network interfaces, either wired or wireless, to provide for enhanced security, monitoring and traffic shaping and management.

### Example Embodiments of Methods for Acquiring and Analyzing Athletic Movement Data

According to one aspect of the embodiment methods of the present disclosure, certain characteristics of an athletic movement can be sensed by a sensor device, such as a force plate, and processed by a local computing device. From the processed sensor data, as well as population data in a database, normalized scores can be determined. In many of the embodiment methods described herein, the characteristics of the athletic movement being sensed can be shown to include statistical indicia of reliability using the Cronbach alpha test. The Cronbach alpha test is a measure of reliability, based on the equation shown below, and the Cronbach alpha score can theoretically be between 0 and 1, with a higher number being more desirable.

$$α = \frac{K}{K - 1} ( 1 - \frac{{\sum }_{i = 1}^{K} {σ}_{{X}_{i}}^{2}}{{σ}_{Y}^{2}} )$$

Generally speaking, a Cronbach score of greater than 0.7 is considered acceptable; a score greater than 0.8 is considered good reliability; and a score greater than 0.9 is considered excellent reliability. Many medical and research professionals require an assessment to have a Cronbach alpha score of at least 0.7 to be acceptable.

According to one embodiment of the present disclosure, based on the movement of a center of pressure during a balance pose, the resultant sway velocity of a user can be determined to assess the static stability of the upper and lower body extremities. As shown in the below tables, using the Cronbach alpha test for a sample of athletes, the resultant sway velocity tests resulted in Cronbach alpha scores of at least approximately 0.80.

Sway Trials (Lower, Left)

| Sample Size | 1732 |
|---|---|
| Cronbach's Alpha Body Weight | .9998 |
| Cronbach's Alpha FRE Int 1 | .8795 |
| Cronbach's Alpha FRE Int 2 | .8095 |
| Cronbach's Alpha Sway Velocity | .8176 |
| Cronbach's Alpha Body Weight/ | .8969 |
| Sway Velocity | |
| Cronbach's Alpha Med. Lat. Sway | .7018 |
| Velocity Int 1 | |
| Cronbach's Alpha Med. Lat. Sway | .7248 |
| Velocity Int 2 | |
| Cronbach's Alpha Ant Post Sway | .7349 |
| Velocity Int 1 | |
| Cronbach's Alpha Sway Velocity | .7358 |
| Int 2 | |

Sway Trials (Lower, Right)

| Sample Size | 1543 |
|---|---|
| Cronbach's Alpha Body Weight | .9991 |
| Cronbach's Alpha FRE Int 1 | .8684 |
| Cronbach's Alpha FRE Int 2 | .8187 |
| Cronbach's Alpha Sway Velocity | .7859 |
| Cronbach's Alpha Body Weight/ | .9028 |
| Sway Velocity | |
| Cronbach's Alpha Med. Lat. Sway | .6746 |
| Velocity Int 1 | |
| Cronbach's Alpha Med. Lat. Sway | .7154 |
| Velocity Int 2 | |
| Cronbach's Alpha Ant Post Sway | .6884 |
| Velocity Int 1 | |
| Cronbach's Alpha Ant Post Sway | .6731 |
| Velocity Int 2 | |

Sway Trials (Upper, Right)

| Sample Size | 1619 |
|---|---|
| Cronbach's Alpha Body Weight | .9940 |
| Cronbach's Alpha FRE Int 1 | .9635 |
| Cronbach's Alpha FRE Int 2 | .9211 |
| Cronbach's Alpha Sway Velocity | .9226 |
| Cronbach's Alpha Body Weight/ | .8892 |
| Sway Velocity | |
| Cronbach's Alpha Med. Lat. Sway | .9050 |
| Velocity Int 1 | |
| Cronbach's Alpha Med. Lat. Sway | .8620 |
| Velocity Int 2 | |
| Cronbach's Alpha Ant Post Sway | .9377 |
| Velocity Int 1 | |
| Cronbach's Alpha Sway Velocity | .7351 |
| Int 2 | |

Sway Trials (Upper, Left)

| Sample Size | 1683 |
|---|---|
| Cronbach's Alpha Body Weight | .9958 |
| Cronbach's Alpha FRE Int 1 | .9488 |
| Cronbach's Alpha FRE Int 2 | .9317 |
| Cronbach's Alpha Sway Velocity | .9521 |
| Cronbach's Alpha Body Weight/ | .9227 |
| Sway Velocity | |
| Cronbach's Alpha Med. Lat. Sway | .9376 |
| Velocity Int 1 | |
| Cronbach's Alpha Med. Lat. Sway | .9377 |
| Velocity Int 2 | |
| Cronbach's Alpha Ant Post Sway | .9483 |
| Velocity Int 1 | |
| Cronbach's Alpha Sway Velocity | .8390 |
| Int 2 | |

According to another embodiment of the present disclosure, a time to stabilize to within a predetermined percentage of a user's reference weight and a peak force generated from a user jumping onto a force plate on one leg can be determined to assess the dynamic stability of the user. Using the Cronbach alpha test for a sample of athletes, the measure of reliability for time to stabilize values for left and right legs during the assessment was 0.835 for left and 0.772, respectively. The reliability for the peak landing force during the assessment was 0.976 for left and 0.978 for right, respectively.

Example embodiment methods for acquiring and analyzing athletic movement data will now be described.

### Example Embodiments of Methods for Assessing Static Stability

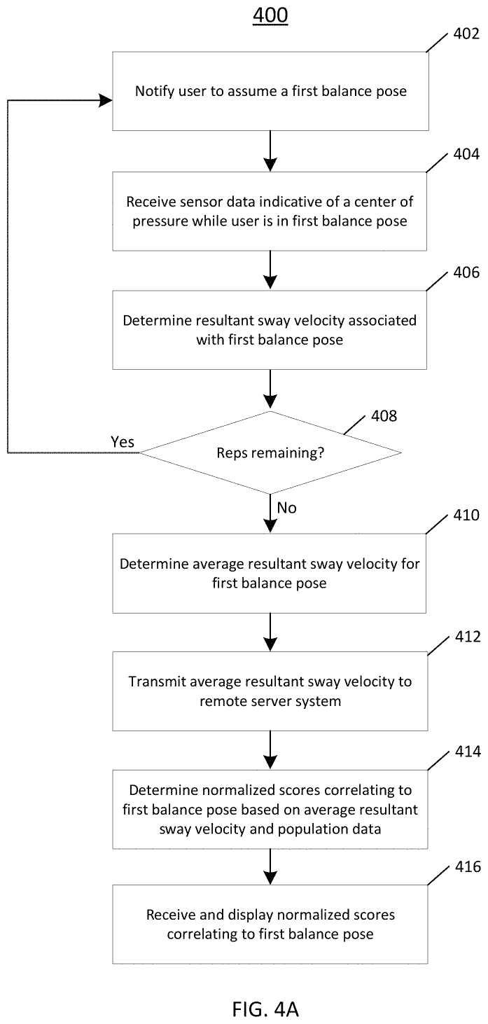

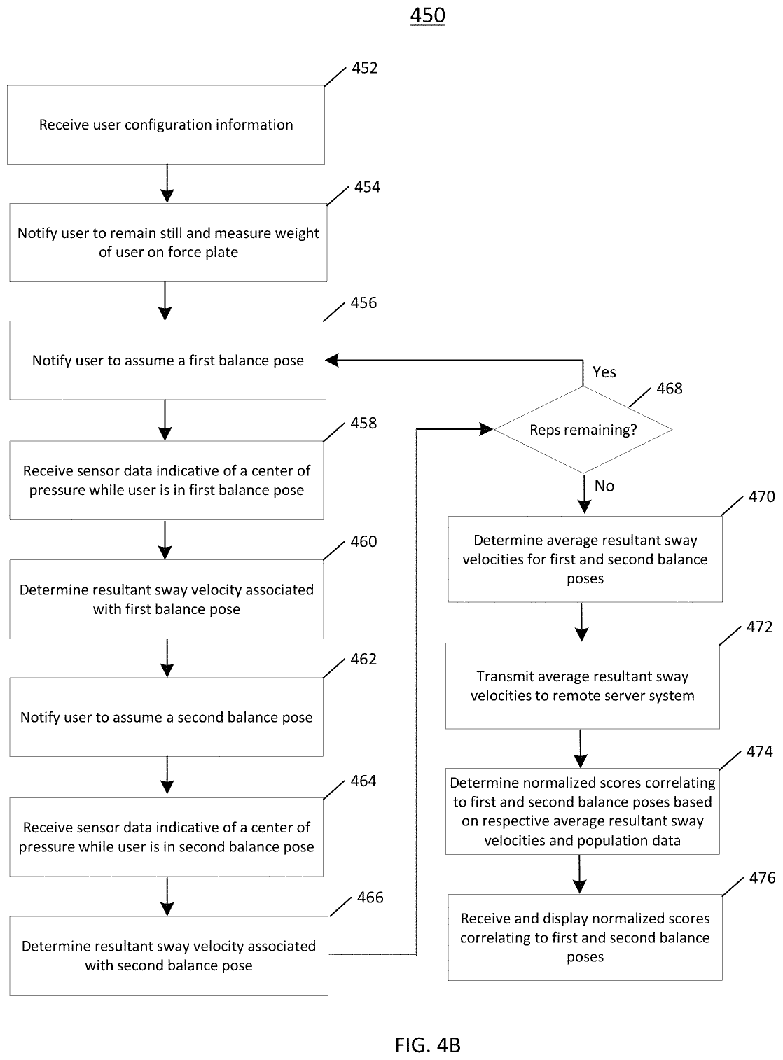

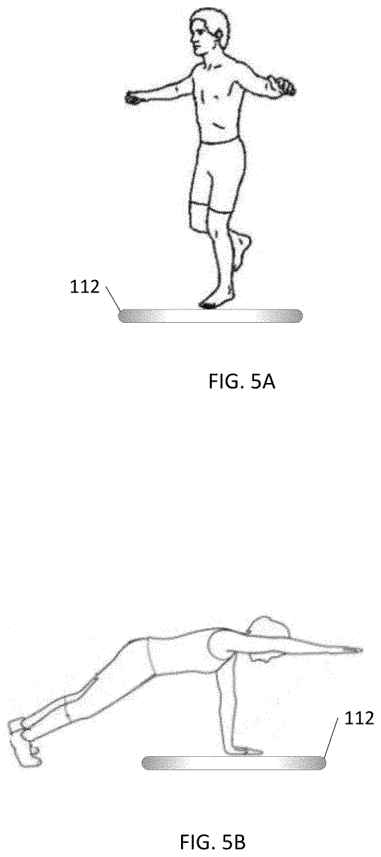

Referring to FIG. 4A, a flow diagram is provided, depicting an overview of an example embodiment of a method 400 for assessing a user's static stability. Those of skill in the art will understand that the method steps disclosed herein can comprise instructions stored in memory of the local or mobile computing device, and that the instructions, when executed by the one or more processors of the local or mobile computing device, can cause the one or more processors to perform the steps disclosed herein. As shown at the top of FIG. 4A at Step 402, a visual or audio notification is first outputted by the local or mobile computing device instructing the user to assume a first balance pose. In some embodiments, prior to Step 402, the user can optionally wear a blindfold. Upon receiving the notification, the user assumes the first balance pose. According to some embodiments in which the static stability of the lower body is being assessed, the first balance pose can comprise the user balancing upon one leg on force plate 112 while maintaining the other leg in a raised position (as shown in FIG. 5A). According to other embodiments in which the static stability of the upper body is being assessed, the first balance pose can comprise the user balancing upon one hand on the force plate while maintaining a plank position (as shown in FIG. 5B).

At Step 404, while the user is in the first balance pose, the local or mobile computing device receives sensor data from the sensor device for a predetermined duration of time (e.g., 20 seconds), wherein the sensor data is indicative of a center of pressure. In some embodiments, the center of pressure can be displayed in real-time on a display of a local or mobile computing device. In other embodiments, the center of pressure can be visually displayed on the local or mobile computing device as a two-dimensional displacement graph. At Step 406, based on the displacement of the center of pressure during the predetermined duration of time, a resultant sway velocity is determined for the first balance pose.

At Step 408, if it is determined that additional repetitions are required, the method returns to Step 402, and a visual or audio notification is outputted by the local computing device instructing the user to assume the first balance pose. In some embodiments, a rest period (e.g., 10 seconds) can be interposed after Step 408, during which the user can release from the first balance pose for a short period of time before being notified to return to the first balance pose at Step 402. If it is determined that no repetitions are remaining at Step 408, then the local or mobile computing device determines an average resultant sway velocity for the first balance pose based on the resultant sway velocities acquired during the repetitions. At Step 412, the average resultant sway velocity is transmitted to the remote server system. In some embodiments, an authentication step can be interposed after Step 410, prior to transmission, in order to ensure that the local or mobile computing device is authorized to transmit data to the remote server system. In some embodiments, the authentication step can be manual, such as requiring the user to input a password at the local or mobile computing device. In other embodiments, the authentication step can be automated through a public or private key exchange.

Referring still to FIG. 4A, at Step 414, one or more normalized scores can be determined based at least in part on: (1) the value of the average resultant sway velocity of the user in the first balance pose, and (2) the mean resultant sway velocity correlating to the first balance pose for a predetermined population of athletes stored in a database residing on, or in communication with, the remote server system. In some embodiments, the predetermined population of athletes can comprise the entire population of athletes for which relevant data is stored in the database. In other embodiments, the predetermined population of athletes can comprise a subset of the entire population of athletes in the database. For example, the normalized scores can be based at least in part on (1) the value of the average resultant sway velocity of the user in the first balance pose, and (2) the mean resultant sway velocity correlating to the first balance pose for athletes stored in the database having the same preferred sport as the user. Other subsets of athletes can include gender, body weight range, age range, injury type, position within a preferred sport. Those of skill in the art will appreciate that these examples are not meant to be exhaustive, and that other subsets of athletes within the database are fully within the scope of the present disclosure. In addition, in many of the embodiments of the present disclosure, the determination of the normalized scores can be performed by the one or more processors of the remote server system by either of the front-end server or the back-end server. In other embodiments, however, the determination of the normalized scores can be performed elsewhere, such as, for example, a local server system (as shown in FIG. 1), or on the local or mobile computing device itself.

At Step 416, the normalized scores are received by the local or mobile computing device and can be displayed in a graphical user interface. As described below with respect to FIGS. 8A to 8C, in some embodiments, the graphical user interface can comprise a bar chart depicting each normalized score as a vertical or horizontal bar. In other embodiments, the graphical user interface can comprise a line plot depicting one or more normalized scores over time.

Turning to FIG. 4B, another flow diagram is provided, depicting an overview of another example embodiment of a method 450 for assessing a user's static stability. As shown at the top of FIG. 4B, at Step 452, user configuration information is inputted into the local computer device. The user configuration information can include, for example, blindfold setting (i.e., indicating whether the user will wear a blindfold during the assessment), a number of repetitions setting (e.g., four repetitions), a repetition duration setting (e.g., 30 seconds), and an upper or lower extremity setting (i.e., indicating whether the user is assessing the static stability of the user's upper body or the user's lower body). In some embodiments, if the blindfold setting is enabled, prior to Step 454, the user can wear a blindfold over the eyes during the assessment. At Step 454, a visual or audio notification is outputted by the local computing device instructing the user to remain still while the sensor device measures the user's weight. At Step 456, a visual or audio notification is outputted by the local or mobile computing device instructing the user to assume a first balance pose. Similar to method 400, in some embodiments, the first balance pose can comprise the user balancing upon one leg on the force plate if the lower body's static stability is being assessed. In other embodiments, the first balance pose can comprise the user balancing on one hand on the force plate while maintaining a plank position if the upper body's static stability is being assessed.

At Step 458, while the user is in the first balance pose, the local or mobile computing device receives sensor data from the sensor device for a predetermined duration of time (e.g., 20 seconds), wherein the sensor data is indicative of a center of pressure. In some embodiments, the center of pressure can be displayed in real-time on the display of local computing device. In other embodiments, the center of pressure can be visually displayed on the local computing device as a two-dimensional displacement graph. At Step 460, based on the displacement of the center of pressure during the predetermined duration of time, a resultant sway velocity is determined for the first balance pose. In some embodiments, Step 460 can include a rest period during which the user can release from the first balance pose for a short period of time (e.g., 10 seconds) before proceeding to Step 462.

At Step 462, a visual or audio notification is outputted by the local computing device instructing the user to assume a second balance pose. In some embodiments where the upper body's static stability is being assessed, assuming the second balance pose can comprise the user alternating from balancing on the right hand on the force plate to balancing on the left hand on the force plate, while in the plank position. Similarly, in other embodiments where the lower body's static stability is being assessed, assuming the second balance pose can comprise the user alternating from balancing on the right leg on the force plate to balancing on the left leg on the force plate, while maintaining the other leg in a raised position. At Step 464, while the user is in the second balance pose, the local computing device receives sensor data from the sensor device for a predetermined duration of time (e.g., 20 seconds), wherein the sensor data is indicative of a center of pressure. At Step 466, based on the displacement of the center of pressure during the predetermined duration of time, a resultant sway velocity is determined for the second balance pose.

At Step 468, if it is determined that additional repetitions are required, the method returns to Step 456, and a visual or audio notification is outputted by the local computing device instructing the user to assume the first balance pose. In some embodiments, another rest period can be interposed after Step 468 during which the user can release from the second balance pose for a short period of time (e.g., 10 seconds) before being notified to return to the first balance pose at Step 456.

If it is determined that no repetitions are remaining at Step 468, then the local computing device determines average resultant sway velocities for each of the first and second balance poses at Step 470 based on the resultant sway velocities acquired during the repetitions. At Step 472, the average resultant sway velocities are transmitted to the remote server system. In some embodiments, an authentication step can be interposed after Step 470, prior to transmission, in a manner similar to method 400 described above.

Referring still to FIG. 4B, at Step 474, normalized scores correlating to the first and second balance poses can be determined based at least in part on: (1) the values of the average resultant sway velocities of the user in the first and second balance poses, and (2) the mean resultant sway velocities correlating to the first and second balance poses for a predetermined population of athletes stored in a database residing on, or in communication with, the remote server system. Similar to method 400, the predetermined population of athletes can comprise the entire population of athletes for which relevant data is stored in the database. In other embodiments, the predetermined population of athletes can comprise a subset of the entire population of athletes in the database, wherein the subsets can include, for example, gender, body weight range, age range, injury type, position within a preferred sport, to name only a few. The determination of normalized scores can be performed by a server of the remote server system, or on a different device such as a local server system (as shown in FIG. 1), or on the local computing device itself.

At Step 476, the normalized scores are received by the local or mobile computing device and can be displayed in a graphical user interface. As described below with respect to FIGS. 8A to 8C, in some embodiments, the graphical user interface can comprise a bar chart depicting each normalized score as a vertical or horizontal bar. In other embodiments, the graphical user interface can comprise a line plot depicting one or more normalized scores over time.

FIGS. 5A and 5B are pictorial diagrams depicting certain steps of methods 400 and 450, as described above. FIG. 5A is a pictorial diagram depicting a user in a first balance pose in which the static stability of the user's lower extremity is being assessed. As can be seen in the diagram, user is balancing upon the left leg on force plate 112, while maintaining the right leg in a raised position such that all weight is resting on the left leg. According to method 450 (FIG. 4B), at Step 462, the user will subsequently alternate sides such that the user is balancing upon the right leg on the force plate while maintaining the left leg in a raised position such that all weight is resting on the right leg. FIG. 5B is a pictorial diagram depicting a user in a first balance pose in which the static stability of the user's upper extremity is being assessed. As can be seen in the diagram, user is balancing upon the left hand on force plate 112 while maintaining a plank position such that all weight is resting on the left hand. Again, according to method 450 (FIG. 4B), at Step 462, the user will subsequently alternate sides such that the user is balancing upon the right hand on the force plate while maintaining a plank position such that all weight is resting on the right hand. Although FIGS. 5A and 5B depict specific balance poses, these poses are meant to be illustrative and non-exclusive. Indeed, those of skill in the art will appreciate that other balance poses in which a user places all or most of the user's body weight upon a portion of the body, which is then positioned on a sensor device capable of measuring a center of pressure, are fully within the scope of the present disclosure.

### Example Embodiments of Methods for Assessing Dynamic Stability

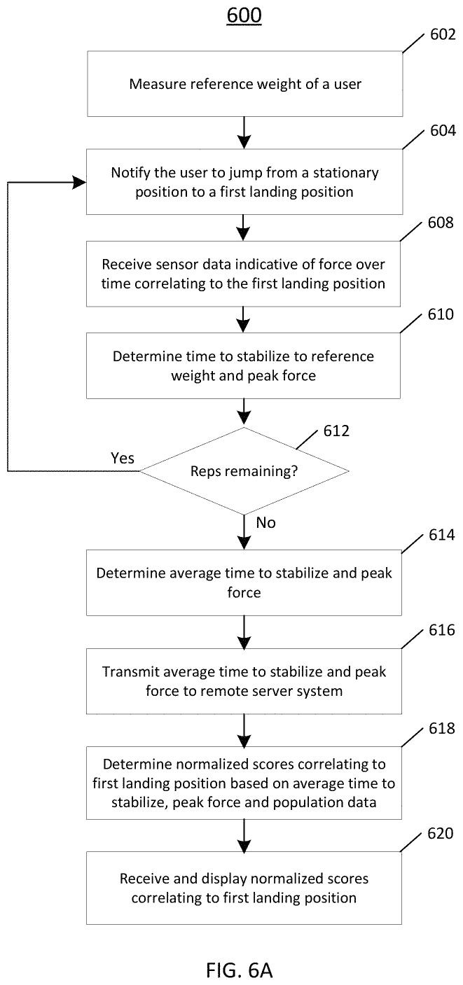

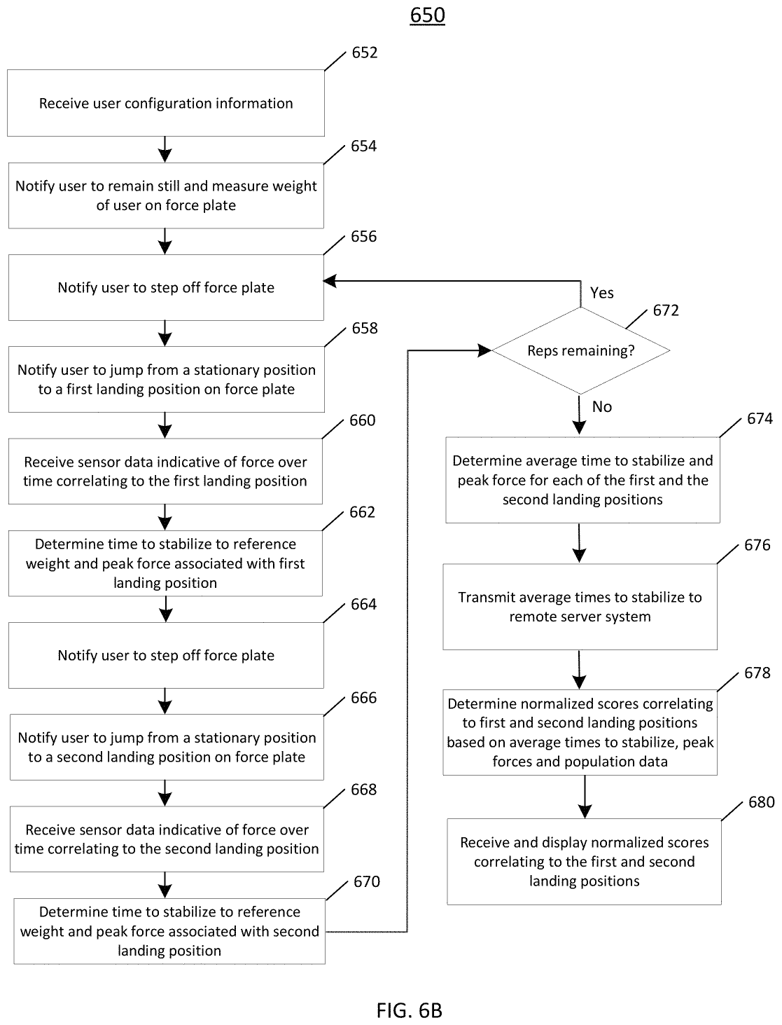

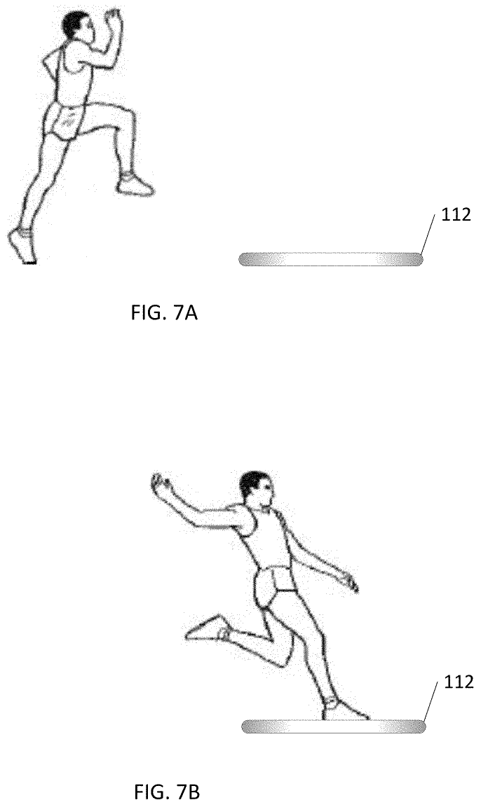

FIG. 6A is a flow diagram depicting an overview of an example embodiment of a method 600 for assessing a user's dynamic stability. Those of skill in the art will understand that the method steps disclosed herein can comprise instructions stored in memory of the local or mobile computing device, and that the instructions, when executed by the one or more processors of the local or mobile computing device, can cause the one or more processors to perform the steps disclosed herein. As shown at the top of FIG. 6A, the user's reference weight is measured at Step 602. In some embodiments, this can be done by having the user stepping on to the sensor device. In other embodiments, the reference weight can be inputted manually by a user. At Step 604, a visual or audio notification is outputted by the local or mobile computing device instructing the user to jump from a stationary position to a first landing position. According to some of the embodiments disclosed herein, the user can begin this step from a stationary position approximately three to five feet away from the center of the sensor device, i.e., the force plate. The distance between the user and the sensor device can be adjusted depending on the circumstances, such as a user's physical limitations. The user subsequently jumps from the stationary position onto the sensor device to a first landing position, wherein the first landing position comprises the user landing on the force plate on one leg, and balancing upon the leg on the force plate while maintaining the other leg in a raised position (as shown in FIGS. 7A and 7B).

At Step 608, while the user is in the first landing position, the local computing device receives sensor data from the sensor device, wherein the sensor data is indicative of the force generated by the user as a function of time.

At Step 610, a time to stabilize value can be determined based on the received sensor data, wherein the time to stabilize can comprise the time elapsed before the force generated by the user while in the first landing position stabilizes to a predetermined percentage of the user's reference weight. For example, in some embodiments, the predetermined percentage can be 5% of the user's reference weight. In other embodiments, the predetermined percentage can be 2% of the user's reference weight. Other predetermined percentages can be used and are fully within the scope of the present disclosure. Additionally, a peak force measured during the time to stabilize can also be determined. According to one aspect of some embodiments, the time to stabilize value can be weighted, normalized or otherwise adjusted according to the peak force associated with the landing position. For example, in some embodiments, a first user that lands with greater force on the force plate will generate a larger peak force than a second user that lands with a smaller force on the force plate. Assuming that the times to stabilize are equal for both users, the time to stabilize of the first user can be adjusted downward by a predetermined factor in order to compensate for the first user's greater peak force.

At Step 612, if it is determined that additional repetitions are required, the method returns to Step 604, and a visual or audio notification is outputted by the local or mobile computing device instructing the user to jump from the stationary position to the first landing position. In some embodiments, a rest period can be interposed after Step 612, during which the user can rest and recover from the previous jump for a short period of time (e.g., 10 seconds) before being notified to perform the jump again at Step 604.

If it is determined that no repetitions are remaining then, at Step 614, the local or mobile computing device determines an average time to stabilize and peak force for the first landing position based on the time to stabilize and peak force values acquired during the repetitions. At Step 616, the average time to stabilize and peak force values are transmitted to the remote server system. In some embodiments, an authentication step can be interposed after Step 614, prior to transmission, in order to ensure that the local or mobile computing device is authorized to transmit data to the remote server system. In some embodiments, the authentication step can be manual, such as requiring the user to input a password at the local or mobile computing device. In other embodiments, the authentication step can be automated through a public or private key exchange.

Referring still to FIG. 6A, at Step 618, one or more normalized scores can be determined based at least in part on: (1) the average time to stabilize of the user in the first landing position, and (2) a mean time to stabilize value correlating to the first landing position for a predetermined population of athletes stored in a database residing on, or in communication with, the remote server system. In some embodiments, the predetermined population of athletes can comprise the entire population of athletes for which relevant data is stored in the database. In other embodiments, the predetermined population of athletes can comprise a subset of the entire population. Subsets of athletes can be categorized by gender, body weight range, age range, injury type, and position within a preferred sport. Those of skill in the art will appreciate that these examples are not meant to be exhaustive, and that other subsets of athletes within the database are fully within the scope of the present disclosure. In addition, in many of the embodiments of the present disclosure, the determination of the normalized scores can be performed by the one or more processors of the remote server system by either of the front-end server or the back-end server. In other embodiments, however, the determination of the normalized scores can be performed elsewhere, such as, for example, by a local server system (as shown in FIG. 1), or by the local or mobile computing device itself.

At Step 620, the normalized scores are received by the local or mobile computing device and can be displayed in a graphical user interface. As described below with respect to FIGS. 8A to 8C, in some embodiments, the graphical user interface can comprise a bar chart depicting each normalized score as a vertical or horizontal bar. In other embodiments, the graphical user interface can comprise a line plot depicting one or more normalized scores over time.

Turning to FIG. 6B, a flow diagram is provided, depicting an overview of another example embodiment of a method 650 for assessing a user's dynamic stability. As shown at the top of FIG. 6B, at Step 652, user configuration information is inputted into the local computer device, or in some alternative embodiments, at a mobile computing device (e.g., tablet computer, smart phone, wearable computing device, etc.). The user configuration information can include, for example, a user condition setting (e.g., indicating whether the user is in a Fresh, Fatigued or Primed condition), a number of repetitions setting (e.g., six repetitions), and an upper, lower, left and right extremity setting (e.g., lower-right extremity). At Step 654, a visual or audio notification is outputted by the local computing device instructing the user to remain still while the sensor device measures the user's weight. Subsequently, at Step 656, a visual or audio notification is outputted by the local computing device instructing the user to step off the force plate.

At Step 658, a visual or audio notification is outputted by the local computing device instructing the user to jump from a stationary position to a first landing position. According to some of the embodiments disclosed herein, the user can begin this step from a stationary position approximately three to five feet away from the center of the sensor device, i.e., the force plate. The distance between the user and the sensor device can be adjusted depending on the circumstances, such as the user's physical limitations. The user subsequently jumps from the stationary position onto the sensor device to a first landing position, wherein the first landing position comprises the user landing on the force plate on one leg, and balancing upon the leg on the force plate while maintaining the other leg in a raised position (as shown in FIGS. 7A and 7B).

At Step 660, while the user is in the first landing position, the local computing device receives sensor data from the sensor device, wherein the sensor data is indicative of the force generated by the user as a function of time.

At Step 662, a time to stabilize value can be determined based on the received sensor data, wherein the time to stabilize can comprise the time elapsed before the force generated by the user while in the first landing position stabilizes to a predetermined percentage of the user's reference weight. For example, in some embodiments, the predetermined percentage can be 5% of the user's reference weight. In other embodiments, the predetermined percentage can be 2% of the user's reference weight. Other predetermined percentages can be used and are fully within the scope of the present disclosure. Additionally, a peak force measured during the time to stabilize can also be determined. According to one aspect of some embodiments, the time to stabilize value can be weighted, normalized or otherwise adjusted according to the peak force associated with the landing position. For example, in some embodiments, a first user that lands with greater force on the force plate will generate a larger peak force than a second user that lands with a smaller force on the force plate. Assuming that the times to stabilize are equal for both users, the time to stabilize of the first user can be adjusted downward by a predetermined factor in order to compensate for the first user's greater peak force.

At Step 664, a visual or audio notification is outputted by the local or mobile computing device instructing the user to step off the force plate. In some embodiments, a rest period can be interposed after Step 664, during which the user can rest and recover from the previous jump for a short period of time (e.g., 10 seconds) before proceeding to Step 666.

At Step 666, a visual or audio notification is outputted by the local or mobile computing device instructing the user to jump from a stationary position to a second landing position. Again, the user can begin this step from a stationary position approximately three to five feet away from the center of the sensor device and jumps onto the sensor device to a first landing position, wherein the second landing position. In many embodiments, the second landing position comprises the user landing and balancing on the force plate using the leg opposite to the one used in Step 658. Consequently, the leg which was used to land and balance in Step 658 is maintained in a raised position at Step 666.

At Step 668, while the user is in the second landing position, the local or mobile computing device receives sensor data from the sensor device, wherein the sensor data is indicative of the force generated by the user as a function of time. At Step 670, a time to stabilize value can be determined based on the received sensor data, wherein the time to stabilize can comprise the time elapsed before the force generated by the user while in the second landing position stabilizes to a predetermined percentage of the user's reference weight. Additionally, a peak force measured during the time to stabilize can also be determined, and can also be used to weight, normalize or other adjust the time to stabilize value.

At Step 672, if it is determined that additional repetitions are required, the method returns to Step 656, and a visual or audio notification is outputted by the local or mobile computing device instructing the user to step off the force plate before continuing on to Step 658, in which the user is instructed to jump again from the stationary position to the first landing position. In some embodiments, a rest period can be interposed after Step 656, during which the user can rest and recover from the previous jump for a short period of time (e.g., 10 seconds) before being notified to perform the jump again at Step 658.

If it is determined that no repetitions are remaining then, at Step 674, the local or mobile computing device determines an average time to stabilize and peak force for the first and second landing positions based on the time to stabilize and peak force values acquired during the repetitions. At Step 676, the average time to stabilize and peak force values are transmitted to the remote server system. In some embodiments, an authentication step can be interposed after Step 674, prior to transmission, in order to ensure that the local or mobile computing device is authorized to transmit data to the remote server system. In some embodiments, the authentication step can be manual, such as requiring the user to input a password at the local or mobile computing device. In other embodiments, the authentication step can be automated through a public or private key exchange.

Referring still to FIG. 6B, at Step 678, one or more normalized scores can be determined based at least in part on: (1) average times to stabilize of the user in the first and second landing positions, and (2) mean time to stabilize values correlating to each of the first and second landing positions for a predetermined population of athletes stored in a database residing on, or in communication with, the remote server system. In some embodiments, the predetermined population of athletes can comprise the entire population of athletes for which relevant data is stored in the database, or a subset thereof. In many of the embodiments of the present disclosure, the determination of the normalized scores can be performed by the one or more processors of the remote server system by either of the front-end server of the back-end server. In other embodiments, however, the determination of the normalized scores can be performed elsewhere, such as, for example, by a local server system (as shown in FIG. 1), or by the local or mobile computing device itself.

At Step 680, the normalized scores are received by the local or mobile computing device and can be displayed in a graphical user interface. As described below with respect to FIGS. 8A to 8C, in some embodiments, the graphical user interface can comprise a bar chart depicting each normalized score as a vertical or horizontal bar. In other embodiments, the graphical user interface can comprise a line plot depicting one or more normalized scores over time.

FIGS. 7A and 7B are pictorial diagrams depicting certain steps of methods 600 and 650, in which a user's dynamic stability is assessed. FIG. 7A is a pictorial diagram showing the user in the middle of a jump toward force plate 112, after leaving a stationary position from a location away from force plate 112. Although FIG. 7A shows user with one leg raised, those of skill in the art will recognize that other methods of jumping are within the scope of the present disclosure. FIG. 7B is a pictorial diagram showing the user as he lands on the force plate 112 on his right leg. As further described with respect to FIGS. 6A and 6B, the user will then balance on the right leg on the force plate until the local computing device has determined that the force has stabilized to within a predetermined percentage of the user's body weight. Subsequently, the jump, land and balance steps can be repeated with the opposite leg. Although FIGS. 7A and 7B depict specific jumping and landing positions, these positions are meant to be illustrative and non-exclusive. Indeed, those of skill in the art will appreciate that other jumping and landing positions and techniques (e.g., landing with two feet, landing on a designated portion of a foot, landing on a designated target on the force plate) are fully within the scope of the present disclosure.

### Example Embodiments of Graphical User Interfaces for Displaying Normalized Scores

Described herein are example embodiments of graphical user interfaces for displaying normalized scores of a user performing one or more athletic movements. As described above, a local or mobile computing device coupled to a sensor device, such as a force plate, can be configured to receive sensor data that is indicative of a characteristic of one or more athletic movements performed by a user. From the received sensor data, according to some of the embodiments disclosed herein, the characteristic can be extracted by the local or mobile computing device as processed sensor data. Subsequently, the processed sensor data is transmitted to a remote server system. The remote server system can receive the processed sensor data, generate a normalized score based on the processed sensor data relative to analogous data for a population of athletes stored in a database, and then transmit the normalized score back to the local or mobile computing device for display.

In many of the embodiments of the present disclosure, the normalized scores include T-scores. T-scores enable a user to take a raw value (e.g., the processed sensor data) and transform it into a standardized score that allows the user to contextualize her assessment within a population of relevant athletes. A standardized score is typically determined by using the mean and standard deviation values from the relevant population data, as represented by the following equation:

$$z = \frac{x - μ}{σ}$$

where z is the standard score, x is the raw score (i.e., processed sensor data), p is the mean of the relevant population, and a is the standard deviation of the relevant population. The T-score is a standard z score shifted and scaled to have a mean of 50 and a standard deviation of 10. A standard z score can be converted to a T-score by the following equation:

T=(z×10)+50

In this regard, T-scores are both meaningful and easy to comprehend. Unlike other standardized measures (e.g., z-scores), T-scores are always positive and typically comprise whole integers. In addition, a T-score of over 50 is above average, a T-score of below 50 is below average, and each increment of 10 represents one standard deviation away from the mean value.

Turning to FIG. 8A, a graphical user interface (“GUI”) 820 is provided for displaying normalized scores representing a user's static stability. GUI 820 includes two vertical bars, wherein a first vertical bar 822 represents a T-score correlating to the average resultant sway velocity of the user's left leg, and wherein a second vertical bar 824 represents the T-score correlating to the average resultant sway velocity of the user's right leg. Below the vertical bars, a date 832 is displayed to indicate the date on which the assessment was performed. As seen at the top of GUI 820, numerical representations of the T-scores (826, 828) are also displayed. At the bottom of GUI 820, the user's body weight 830 is displayed for reference. According to the embodiment shown, the user's T-scores for the static stability of the left leg and the right leg are 59 and 55, respectively, which can indicate that both legs are within one standard deviation of the mean static stability for the relevant population of athletes. For ease of reference, the numerical representations of the T-scores (826, 828) can be whole numbers which are always positive. With respect to an injured athlete, for example, these T-scores may provide an indication that the athlete is ready to return to play.

Turning to FIG. 8B, another GUI 840 is provided for displaying normalized scores representing a user's dynamic stability. GUI 840 also includes two vertical bars, wherein a first vertical bar 842 represents the T-score correlating to the average time to stabilize for a user's left leg, and wherein a second vertical bar 844 represents the T-score correlating to the average time to stabilize for a user's right leg. At the top of GUI 840, numerical representations of the T-scores (846, 848) are also displayed. Additionally, GUI 840 depicts reference line 850 at a T-score of 40, along with “Stability” and “Mobility” labels 852 to indicate to the user how to best interpret the result. According to the embodiment shown, the user's T-scores for the dynamic stability of the left leg and the right leg are 51 and 55, respectively, which can indicate that both legs are within one standard deviation of the mean dynamic stability for the relevant population of athletes. Again, for ease of reference, the numerical representations of the T-scores (846, 848) can be whole numbers which are always positive. In a healthy athlete, for example, these T-scores may provide an indication that the athlete is not susceptible to injury with respect to the lower extremity of the body.

Turning to FIG. 8C, another GUI 860 is provided for displaying a plurality of normalized scores representing a user's static stability over a certain time period. GUI 860 displays two plotted lines (862, 864), wherein a first plotted line 862 represents a user's T-scores correlating to the average resultant sway velocity for a user's left leg between May 11, 2015 and Jun. 2, 2015, and wherein a second plotted line 864 represents a user's T-scores correlating to the average resultant sway velocity for a user's right leg in the same time frame. At the bottom of GUI 860, along the bottom of the chart, specific dates 866 for each of the sets of T-scores are displayed. According to the embodiment shown, plotted lines (862, 864) show the user's historical T-scores for the static stability of each leg during a twenty-one day period. For an injured athlete, for example, GUI 860 can provide an indication to the athlete of her progression towards return to play or, similarly, the effectiveness of a certain training program or rehabilitation regime.

Thus, the simple GUIs 820, 840 and 860 can offer easy-to-understand metrics to a user in the context of specific athlete populations, without a need for the user to understand or interpret the underlying and complex measurements acquired by the sensor device. These examples are meant to be illustrative, and not limiting in any sense, as those of skill in the art will readily understand that other types and formats of graphical representations of a user's T-scores are within the scope of the disclosed embodiments.

### Example Embodiments of Methods and Interfaces for Athletic Movement Data Validation

Example embodiments of methods for validating athletic movement data will now be described. Those of skill in the art will understand that the method steps disclosed herein can comprise instructions stored in memory of the local computing device, or in some alternative embodiments, in a mobile computing device or a remote server system, and that the instructions, when executed by the one or more processors, can cause the one or more processors to perform the steps disclosed herein.

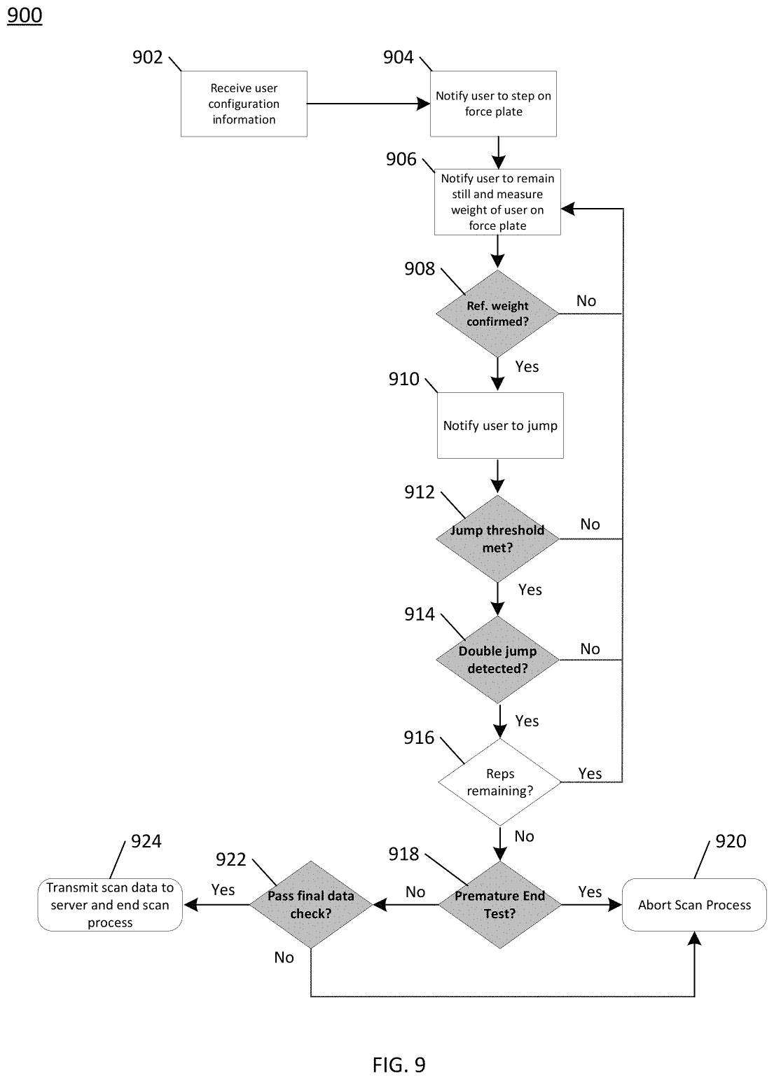

Referring first to FIG. 9, a flow diagram is provided, depicting an overview of an example embodiment of a method 900 for generating an athletic signature for a user, the method including one or more data validation steps (shown as shaded diamonds). At Step 902, user configuration information is received at a local computing system, or in some alternative embodiments, at a mobile computing device (e.g., tablet computer, smart phone, wearable computing device, etc.). The user configuration information can include any one or more of the following settings: a weight tolerance setting, a still criteria setting, a countdown timer setting, an upward movement threshold setting, a jump error settings, a jump height threshold setting, a gender setting, a sport setting, and/or a position within a sport setting. At Step 904, a visual or audio notification is outputted by the local computing device instructing the user to step on the sensor device, i.e., the force plate. At Step 906, a visual or audio notification is outputted by the local computing device instructing the user to remain still while the sensor device measures the user's weight. At Step 908, the measured weight can be validated against a reference weight stored in memory for the particular user. For example, the validation can comprise checking if the measured weight is within a certain predetermined percentage of the stored reference weight for the user. In other embodiments, for example, the validation can comprise checking if the measured weight is within a certain predetermined percentage of the last weight measurement taken for the user. At Step 910, a visual or audio notification is outputted by the local or mobile computing device instructing the user to perform a vertical jump.

At Step 912, a determination can be made as to whether the user's jump has met a jump height threshold, which can include either or both of a minimum jump height and a maximum jump height. If the jump height threshold is not met, then the method returns to Step 906, and an audio or visual notification is outputted by the local or mobile computing device instructing the user to remain still while the sensor device measures the user's weight again. If the jump threshold is met, then at Step 914, another determination can be made as to whether a jump error, such as a double jump, has been detected. If a jump error has been detected, then the method returns to Step 906. If no jump error is detected, then at Step 916, a determination is made as to whether any repetitions remain. At Step 918, a determination is made whether the method has ended prematurely. A premature end may be determined, for example, if no repetitions are remaining, but there is an insufficient amount of data generated. A premature end may also be determined, for example, if the user steps off the sensor data and does not return before a timeout countdown has expired.

Referring still to FIG. 9, if no premature end is determined, then at Step 922, a final data check is performed. In some of the embodiments of the present disclosure, the final data check can include one or more steps taken to ensure that the file to be transmitted to the remote server system is not corrupted (e.g., CRC checksum). If the final data check is passed, then at Step 924, the data is transmitted to the remote server system.

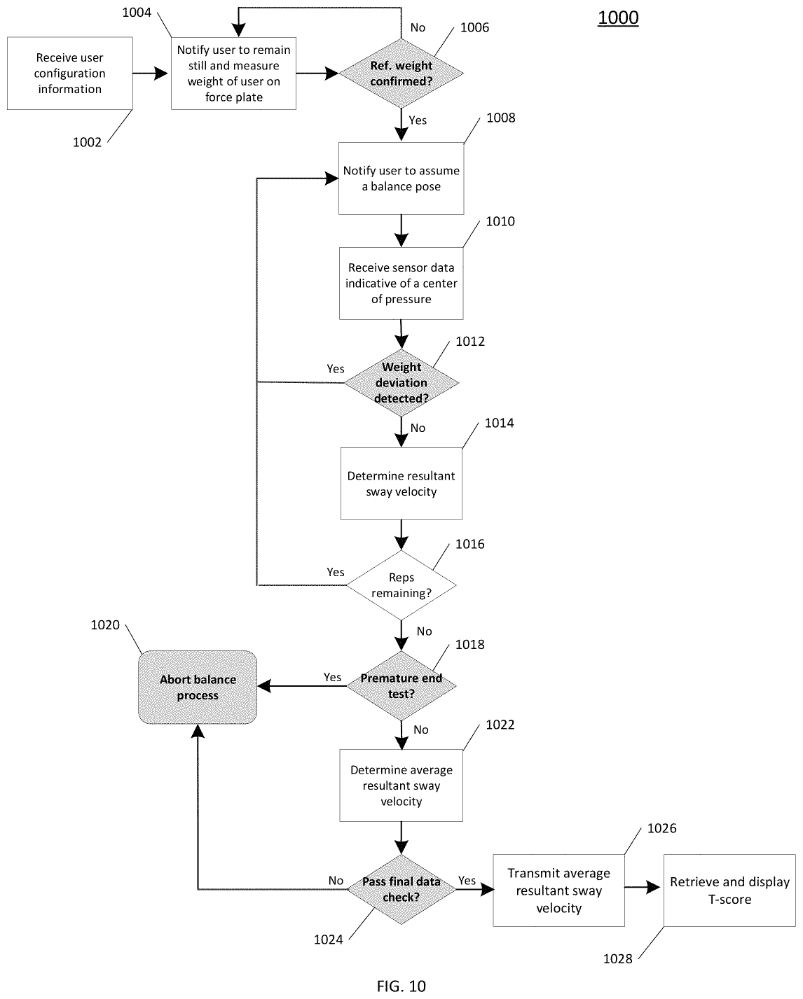

Referring to FIG. 10, a flow diagram is provided, depicting an overview of an example embodiment of a method 1000 for assessing a user's static stability, the method including one or more data validation steps (shown as shaded diamonds). Those of skill in the art will appreciate that one or more of the steps of either methods 400 and 450, described earlier with respect to FIGS. 4A and 4B, are freely combinable with the steps of method 1000. Additionally, method 1000 can include Step 1006, wherein a weight confirmation is determined. The weight confirmation can include one or more steps to determine whether a measured weight of a user falls within a predetermined percentage of a stored reference weight for the same user. In some embodiments, the weight confirmation can comprise one or more steps to determine whether the measured weight of the user is within a predetermined percentage of a recent prior measured weight of the user. Method 1000 can also include Step 1012, in which a weight deviation is determined. In many of the embodiments disclosed herein, the weight deviation determination comprises determining whether a user has inadvertently stepped off the plate or lost his or her balance during the measurement.

Referring still to FIG. 10, at Step 1018, a determination can be made whether the method has ended prematurely. A premature end can be determined, for example, if no repetitions are remaining, but there is an insufficient amount of data generated. A premature end may also be determined, for example, if the user steps off the sensor data and does not return before a timeout countdown has expired. At Step 1024, a final data check is performed. In some of the embodiments of the present disclosure, the final data check can include one or more steps taken to ensure that the file to be transmitted to the remote server system is not corrupted (e.g., CRC checksum). If the final data check is passed, then at Step 1026, the data is transmitted to the remote server system.

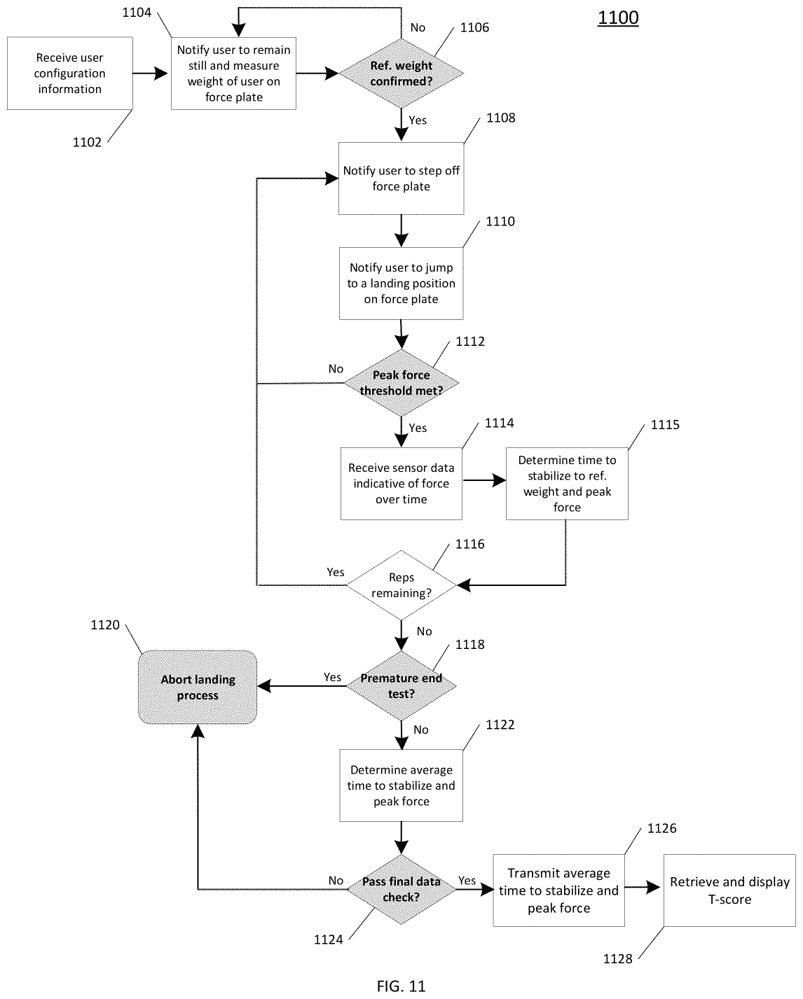

Referring to FIG. 11, a flow diagram is provided, depicting an overview of an example embodiment of a method 1100 for assessing a user's dynamic stability, the method including one or more data validation steps (shown as shaded diamonds). Those of skill in the art will appreciate that one or more of the steps of either methods 600 and 650, described earlier with respect to FIGS. 6A and 6B, are freely combinable with the steps of method 1100. Additionally, method 1100 can include Step 1106, wherein a weight confirmation is determined. The weight confirmation can include one or more steps to determine whether a measured weight of a user falls within a predetermined percentage of a stored reference weight for the same user. In some embodiments, the weight confirmation can comprise one or more steps to determine whether the measured weight of the user is within a predetermined percentage of a recent prior measured weight of the user. Method 1100 can also include Step 1112, in which it is determined whether a peak force threshold has been met. In many of the embodiments disclosed herein, the peak force threshold determination comprises determining whether a user has landed on a force plate, for example, with sufficient force. In some embodiments, a maximum peak force value can also be utilized to ensure that a user does not land on a force plate with excess force.

Referring still to FIG. 11, at Step 1118, a determination can be made whether the method has ended prematurely. A premature end may be determined, for example, if no repetitions are remaining, but there is an insufficient amount of data generated. A premature end may also be determined, for example, if the user steps off the sensor data and does not return before a timeout countdown has expired. At Step 1124, a final data check is performed. In some of the embodiments of the present disclosure, the final data check can include one or more steps taken to ensure that the file to be transmitted to the remote server system is not corrupted (e.g., CRC checksum). If the final data check is passed, then at Step 1126, the data is transmitted to the remote server system.

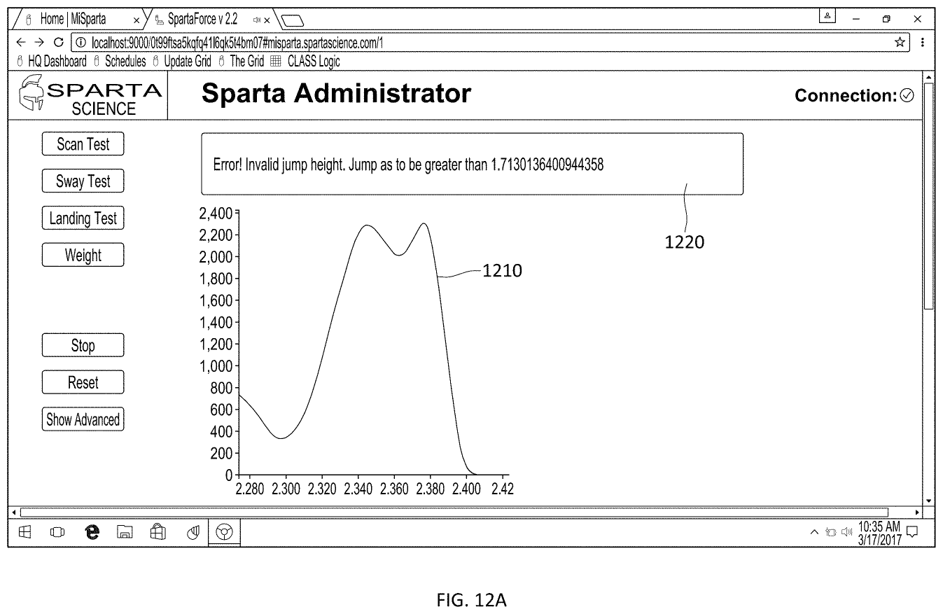

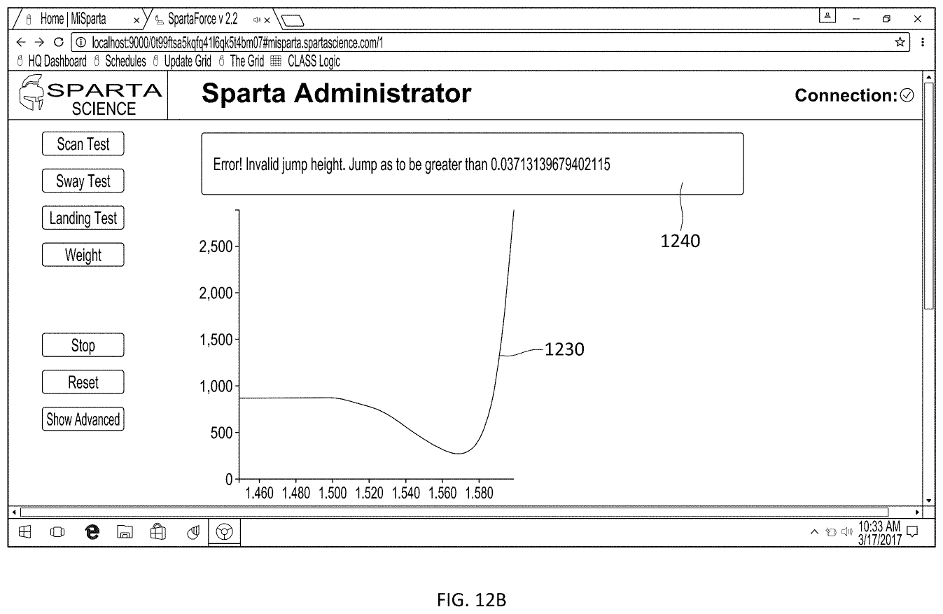

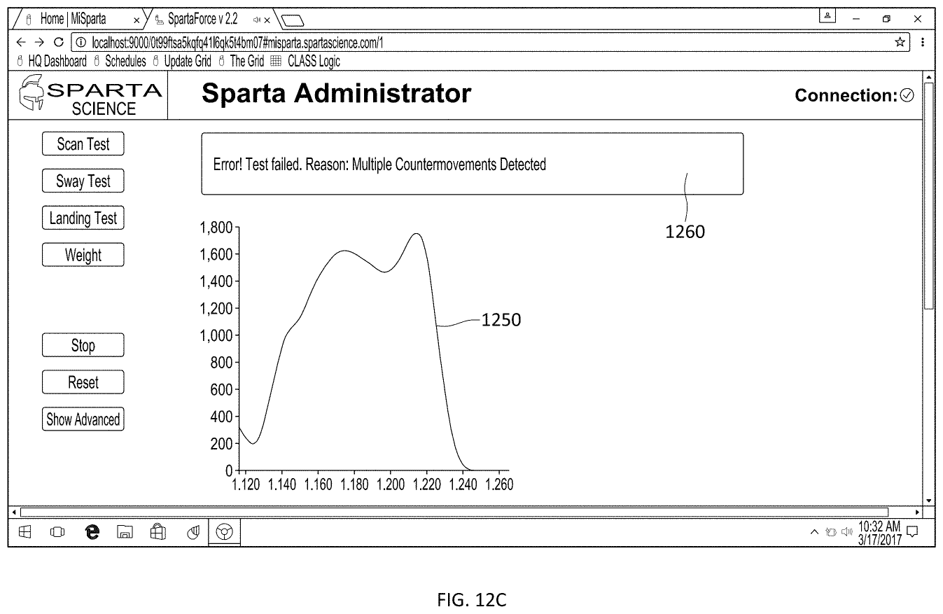

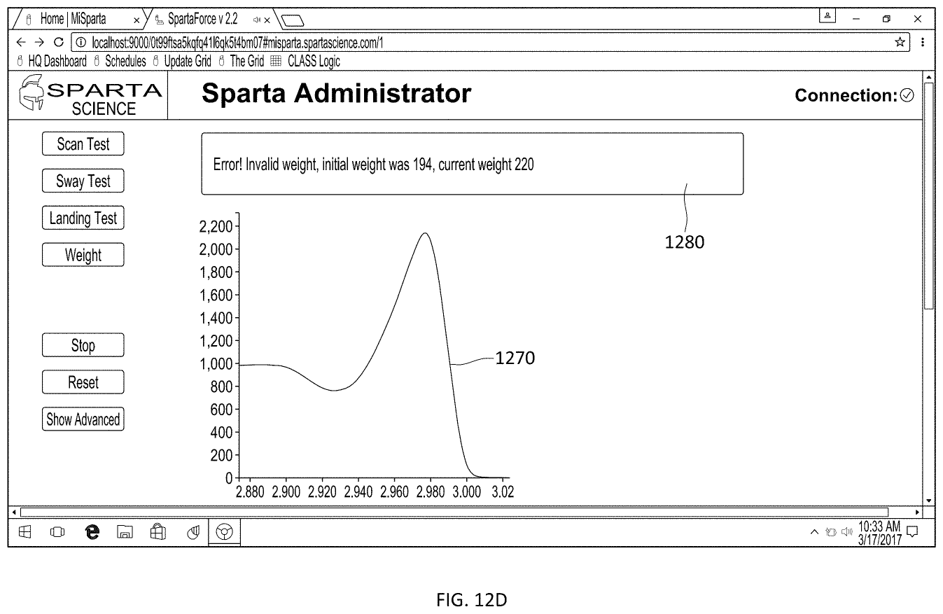

FIGS. 12A to 12D are example embodiments of graphical user interfaces (“GUIs”) for displaying various data validation notifications consistent with the method steps described with respect to FIGS. 9, 10 and 11. In particular, and in accordance with the steps of example embodiment method 900 that are described with respect to FIG. 9, a visual notification is depicted in FIG. 12A showing a graphical representation 1210 of a user who has not jumped to a sufficient height to meet a jump threshold. A textual message 1220 is displayed, stating “Error! Invalid jump height. Jump has to be greater than 1.7130.” Similarly, with respect to FIG. 12B, a visual notification is shown for a graphical representation 1230 of a user who has not met the jump threshold. In the depicted embodiment, the visual notification can display in response to a user jumping but failing to land on the force plate. A textual message 1240 is displayed, stating “Error! Invalid jump height. Jump has to be greater than 0.0371,” indicating that the user has not landed on the force plate.

FIG. 12C depicts a visual notification showing a graphical representation 1250 where it has been determined that the user has performed a “double jump.” A textual message 1260 is displayed, stating “Error! Test failed. Reason: Multiple Countermovements Detected.” In some embodiments, a “double jump” can be determined if the user fails to perform a single vertical jump with a single countermovement prior to the jump. For example, a “double jump” may be detected if the user has swung his or her arms multiple times before thrusting upward into a vertical jump. As another example, a “double jump” may be detected if the user performs one or more bouncing motions before thrusting upward into a vertical jump.

FIG. 12D depicts a visual notification showing a graphical representation 1270 where it has been determined that the weight measure is not within a predetermined percentage of a stored reference weight. A textual message 1280 is displayed, stating “Error! Invalid weight, initial weight was 194, current weight 220.” In some embodiments, failure of the weight confirmation process can be caused one or more of the following reasons: user entering the incorrect identification information; user not positioned on the force plate correctly during the weight measurement process; or force plate may need to be re-calibrated.

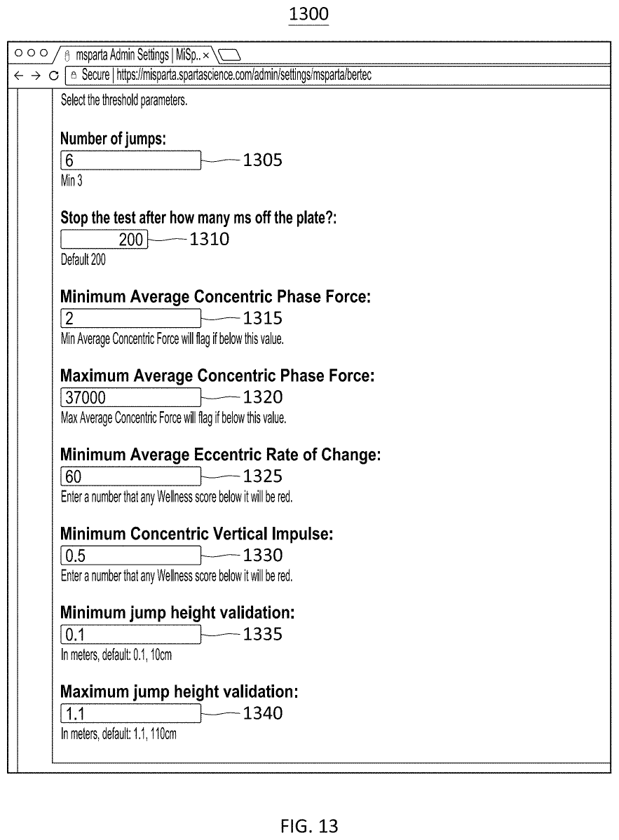

FIG. 13 is a graphical user interface 1300 depicting various user configuration settings that can be inputted into either a local computing device or a mobile computing device which is communicatively coupled to a sensor device, i.e., a force plate. In accordance with the example embodiment methods described herein, the user configuration settings can include one or more of the following: number of jumps setting 1305, premature end test timer 1310, minimum average concentric phase force (i.e., minimum peak force) setting 1315, maximum average concentric phase force (i.e., maximum peak force) setting 1320, minimum average eccentric rate of change setting 1325, minimum concentric vertical impulse 1330, minimum jump height setting 1335, and a maximum jump height setting 1340. Although not shown, other user configuration settings can include one or more of the following: a weight tolerance setting, a still criteria setting, a countdown timer setting, an upward movement threshold setting, a jump error settings, a gender setting, a sport setting, and/or a position within a sport setting. Those of skill in the art will appreciate that any combination of the above settings can be utilized for any of the embodiment methods disclosed herein. Additionally, those of skill in the art will understand that the GUIs and settings described herein can comprise instructions stored in memory of the local computing device, a mobile computing device, a local server system or a remote server system, and that the instructions, when executed by one or more processors, cause the one or more processors to generate and output the described GUIs and user configuration interfaces described herein.

It should also be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. A method for assessing a user's static stability, the method comprising:
notifying a user to assume a first balance pose on a force plate;
receiving a first set of sensor data indicative of a center of pressure, wherein the first set of sensor data is generated by the force plate while the user is in the first balance pose;
determining, based on the first set of sensor data, a resultant sway velocity associated with the first balance pose;
transmitting the resultant sway velocity associated with the first balance pose to a remote server system; and
receiving from the remote server system and displaying on a local computing device one or more T-scores correlating to the resultant sway velocity associated with the first balance pose.

2. The method of claim 1, further comprising:
after receiving the first set of sensor data, notifying the user to assume a second balance pose on the force plate;
receiving a second set of sensor data indicative of a center of pressure, wherein the second set of sensor data is generated by the force plate while the user is in the second balance pose;
determining, based on the second set of sensor data, a resultant sway velocity associated with the second balance pose;
transmitting the resultant sway velocity associated with the second balance pose to a remote server system; and
receiving from the remote server system and displaying on a local computing device one or more T-scores correlating to the resultant sway velocity associated with the second balance pose.

3. The method of claim 1, wherein the first balance pose comprises the user balancing upon a first leg on the force plate while maintaining a second leg in a raised position.

4. The method of claim 3, wherein the second balance pose comprises the user balancing upon the second leg on the force plate while maintaining the first leg in a raised position.

5. The method of claim 1, wherein the first balance pose comprises the user balancing upon a first hand on the force plate while maintaining a plank position.

6. The method of claim 5, wherein the second balance pose comprises the user balancing upon a second hand on the force plate while maintaining a plank position.

7. The method of claim 1, wherein the first set of sensor data includes sensor data generated by the force plate in response to a predetermined number of repetitions performed by the user assuming the first balance pose on the force plate.

8. The method of claim 7, wherein the resultant sway velocity associated with the first balance pose is based at least in part on an average value of the resultant sway velocity for the predetermined number of repetitions performed by the user assuming the first balance pose on the force plate.

9. The method of claim 8, wherein the second set of sensor data includes sensor data generated by the force plate in response to a predetermined number of repetitions by the user assuming the second balance pose on the force plate.

10. The method of claim 9, wherein the resultant sway velocity associated with the second balance pose is based at least in part on an average value of the resultant sway velocity for the predetermined number of repetitions by the user assuming the second balance pose on the force plate.

11. The method of claim 1, wherein the force plate includes one or more piezoelectric sensors.

12. The method of claim 2, further comprising, concurrently displaying, as a vertical bar chart, each of the T-scores correlating to the resultant sway velocity associated with the first balance pose and the second balance pose.

13. The method of claim 1, further comprising, displaying, as a plotted line, a plurality of T-scores correlating to the resultant sway velocity associated with the first balance pose over time.

14. The method of claim 2, further comprising, displaying, as a plotted line, a plurality of T-scores correlating to the resultant sway velocity associated with the second balance pose over time.

15. The method of claim 1, further comprising, selecting, by the remote server system, the one or more T-scores correlating to the resultant sway velocity associated with the first balance pose based on a gender of the user.

16. The method of claim 1, further comprising, selecting, by the remote server system, the one or more T-scores correlating to the resultant sway velocity associated with the first balance pose based on a preferred sport of the user.

17. The method of claim 16, further comprising, selecting, by the remote server system, the one or more T-scores correlating to the resultant sway velocity associated with the first balance pose based on a preferred position within the preferred sport of the user.

18. The method of claim 1, further comprising storing, by the remote server system, the resultant sway velocity associated with the first balance pose in a database.

19. The method of claim 18, further comprising storing, by the remote server system, the resultant sway velocity associated with the second balance pose in the database.

20. The method of claim 1, further comprising displaying on a local computing device an assessment of the user's static stability based on the one or more T-scores correlating to the resultant sway velocity associated with the first balance pose.

21-75. (canceled)
